(19) United States
(12) Patent Application Publication
(10) Pub. No.: US 2008/0188905 A1
(43) Pub. Date: Aug. 7, 2008

(54) Stimulus dose system and method for electroconvulsive therapy

(75) Inventor: Conrad M. Swartz, Springfield, IL (US)

Correspondence Address: HAHN LOESER & PARKS, LLP, One GOJO Plaza, Suite 300, AKRON, OH, 44311-1076, US

(73) Assignee: SOMATICS LLC, Lake Bluff, IL (US)

(21) Appl. No.: 11/670,589

(22) Filed: Feb. 2, 2007

(51) Int. Cl. A61N 1/36

(52) U.S. Cl. 607/45

(57) ABSTRACT

A method of treating a patient includes administering an electroconvulsive therapy (ECT) stimulus to the patient. The current is greater than about 0.9 ampere or 1.0 ampere, or is between about 1.0 and about 2 amperes. The energy delivered is about 100 Joules or less across an impedance of 200 ohms. The stimulus may have a pulsewidth between about 0.5 and about 2.0 msec and/or a frequency in the range of about 10 to about 180 Hz, or the stimulus may have a pulsewidth between about 0.2 and about 0.499 msec and/or a frequency in the range of about 15 to about 300 Hz. An apparatus for ECT comprises a generator adapted to deliver such an electroconvulsive therapy stimulus to a patient. A method of calculating ECT dose comprises multiplying a charge to be delivered by the current (or voltage) to an exponential power between 2 and 4.

### BACKGROUND OF THE INVENTION

The present invention relates to medicine and more particularly to the use of an electrical stimulus applied to the head of the patient in the treatment known as electroconvulsive therapy (ECT).

Electroconvulsive therapy (“ECT”), non-medically called “shock therapy”, is used to treat mood disorders and acute psychoses such as major depression, catatonia and schizophreniform illness. A report of a panel from the National Institute of Mental Health, printed in the journal Science (Jun. 28, 1984, pp. 1510-11) concluded that “not a single controlled study has shown another form of treatment to be superior to ECT in the short-term management of severe depressions.” This panel noted that the complication rate is about 1 in 1700 treatments and that severe and prolonged memory loss is extremely rare and possibly non-existent.

In an ECT treatment two electrodes are applied to the skin of the patient, on the head, and an electric current passes between the electrodes. The current depolarizes brain neurons and triggers a brain seizure event that develops into a generalized convulsive episode that resembles the seizure of generalized epilepsy. This resemblance is by the observable appearance of tonic and clonic muscle movements, by display of characteristic seizure activity on an electroencephalogram, by measurement of release of the hormone prolactin into the bloodstream, and by elevations in the heart rate and blood pressure that persist for about as long as the generalized seizure does.

ECT stimulus generator instruments that are commercially available in the USA provide an electrical stimulus that is a series of rectangular waves each of width 0.25 to 2 msec, commonly called pulses. These instruments deliver between 20 and 280 bipolar pulses per second at a constant current of 0.9 amps or less. The train (or series) of pulses is adjustable from 0.1 to 8.0 seconds in duration.

In treating a patient with medication, the doctor must prescribe the medication dose to be administered. Analogously with ECT treatment, the electrical dose must be prescribed. The ECT stimulus dose has long been stated as its electrical charge in millicoulombs (mC). The charge is equal to the multiplication product of the current, the pulsewidth, and the number of pulses in the stimulus. However, the charge is not a correct expression of the dose. This is because the charge omits the crucial effects of voltage and time. The charge represents only the number of electrons in the stimulus, and therefore cannot distinguish between delivering the stimulus rapidly, with adequately high voltage and current to induce a seizure, or slowly, with inadequate seizure-inducing properties. A charge is appropriate for describing the amount of electricity used in restoring the function of a drained rechargeable automobile battery, that is, “recharging” it. However, it is inappropriate for describing the function of electricity in an ECT stimulus dose.

The voltage of an ECT stimulus should optimally exceed a minimum needed to induce the phenomenon of “neuronal firing,” commonly and approximately referred to as neuronal depolarization. If the voltage does not exceed this minimum it does not induce a seizure, but using the charge as the dosing unit ignores this effect. Most ECT stimulus generators are of constant current design, and for these the minimum voltage translates into a minimum current according to Ohm's Law, that voltage equals current multiplied by impedance.

Expressing the ECT stimulus dose in units of stimulus energy (e.g., joules) is similarly incorrect because stimulus energy represents only the total heat liberated by the electrical stimulus and does not reflect the time taken to release this heat or otherwise deliver the stimulus. It also does not reflect the voltage or the current, or the minimum voltage or current needed.

To accurately reflect seizure-inducing capability, the unit of stimulus dose must be related to the desired result, the induction of a generalized brain seizure. Previously, there was substantial confusion about the formulation of the dose of the ECT stimulus. To illustrate, Andrade (“Quantifying the ECT Dose: The Right Unit Remains Elusive,” J. ECT, 2001, 17:75) mentioned difficulty in understanding how a wider pulsewidth could compensate for a lower frequency or a shorter pulse train duration in the ECT stimulus dose.

A common problem in electroconvulsive therapy occurs when the stimulus does not induce a seizure of good quality, or even any seizure, in contrast to previous treatment sessions with the same patient. In other words, the patient develops resistance to seizure induction. An example of a seizure that is not of good quality is one that shows no tonic-clonic muscle activity. The usual remedy is to increase the stimulus charge, while using the maximum current available from the ECT instrument. Often the maximum charge is reached but still no good seizure can be obtained. This problem tends to occur with older patients, after they have received at least three ECT treatments.

Another tactic is to administer high-dose intravenous caffeine, but studies with laboratory animals suggest that this can injure brain cells in the hippocampus, the memory center of the brain (Enns et al., “Hippocampal Neurons Are Damaged By Caffeine-Augmented Electroshock Seizures,” Biological Psychiatry, 1996, 40: 642-7). It also causes arrhythmias of the heart, increased blood pressure, and increased pulse, each of which poses risks for injuring the heart.

Still another tactic is switching to a nonsedating anesthetic such as etomidate. However, this increases hypertension and tachycardia immediately following the ECT treatment, which risks injuring the heart and takes substantial time from the treating doctors and nurses. Etomidate is more costly than other anesthetics used for ECT. The safest, most efficient, and least expensive method to overcome resistance to seizure induction should be to increase the available dose of the electrical stimulus.

Up to now, the stimulus dose has been increased only by increasing the charge while keeping the current at the instrument's maximum, which is 800 mA for some instruments and 900 mA for others. According to U.S. federal regulations on ECT instruments in the USA and some other countries, the specification of the maximum ECT stimulus allowable is that it has an energy of 100 joules across an impedance of 220 ohms.

This limitation on the energy of the stimulus controls only how much the stimulus can raise the temperature of the head, including the skin, the scalp, the skull and the brain. Thereby it limits only the risk of undesirable temperature increase.

### SUMMARY OF THE INVENTION

The invention identifies for the first time that stimulus energy and its temperature effects are not relevant to ECT seizure induction. The invention allows an increase in the dose relevant to ECT seizure induction without increasing the amount of energy in the stimulus beyond the specified maximum permitted by U.S. federal regulations.

In one aspect, the invention provides a method of treating a patient. The method comprises administering an electroconvulsive therapy (ECT) stimulus to the patient, wherein the current is greater than about 0.9 ampere. In some examples, the current is greater than about 1 ampere. In other examples, the current is between 1 ampere and about 2 amperes. The invention may also include an ECT stimulus where the energy delivered is less than or equal to about 100 Joules across an impedance of 200 ohms. Furthermore, the current may vary by 0.1 ampere or less.

In another aspect, the invention provides an apparatus for electroconvulsive therapy comprising an electroconvulsive therapy stimulus generator, wherein the generator is adapted to deliver an electroconvulsive therapy stimulus to the patient, the stimulus comprising a current greater than about 0.9 ampere. In some examples, the current is greater than about 1 ampere. In other examples, the current is between 1 ampere and about 2 amperes.

An instrument for ECT may display the stimulus dose that is calculated as the product of the charge and an exponential power of the current, which may frequently be the third power, but may also be other powers. This display can be in any observable form, such as light emitting display, liquid crystal display, analog display, cathode ray tube, or paper printout. As explained below, the number of this exponential power can be between 2 and 3 for some electrode placements. Stimulus doses can be multiplied by a constant scaling factor besides unity without departing from the invention.

In still another aspect, the invention provides a method of calculating the dose of an electroconvulsive therapy stimulus to be delivered to a patient, the method comprising multiplying a charge to be delivered by the current to an exponential power, wherein the exponential power is between about 2 and about 4. The calculation may be incorporated into an ECT stimulus generator. The stimulus dose may be set by the operator, where this stimulus dose is calculated as the product of the charge and an exponential power of the current.

### DETAILED DESCRIPTION OF THE INVENTION

To relate and connect electricity in an ECT stimulus to the seizure that it induces requires a reasonable description of the seizure-inducing mechanism that relates to equations of physics. The mechanism begins with the electrical stimulus depolarizing brain neurons, which then causes neuronal firing. In turn, this neuronal firing spreads to other neurons which then depolarize and fire in response. The brain neurons that were depolarized by the electricity are the seizure foci from which the seizure spreads. Such electrically-induced seizures have self-limited duration that is typically in the range of 30-90 seconds. These foci differ from the seizure foci of epilepsy, which are persistent and typically associated with an abnormal microscopic anatomy that represents past injury. The persistence of an epileptic seizure focus allows gradual seizure development over hours to days, which is conspicuously slower than the few seconds taken for the development of the ECT seizure. Here the term “seizure foci” will be taken to have the same meaning as brain neurons that fire in response to direct depolarization by the ECT stimulus. The minimum volume of seizure foci required to induce a generalized brain seizure corresponds to what has been traditionally called the “seizure threshold.” To reach this threshold, the voltage across the electrodes must be high enough to depolarize neurons and cause them to fire and also a sufficient number of electrons (that is, charge) must pass between the electrodes. If the voltage is substantially higher than the minimum, it is consistent with this model, and possible, that the minimum charge can be lower than if the voltage is near the minimum. However, regardless of the charge, a certain minimum voltage is needed to cause neuronal depolarizion and firing.

The invention also includes a set of mathematical equations that corresponds to the mechanism. This starts with identifying reasonable approximations that allow equations to be written. The first approximation is that the electrode sites are separated enough so that each electrode site can be considered as separate from the other. The second approximation is that each electrode site is small compared to the distance between the electrodes and so can be represented by a point. The third approximation is that the brain has the same electrical impedance in all directions throughout; this implies that the voltage drops linearly between the two electrodes and a particular voltage drop corresponds to a particular physical distance. The fourth approximation is that the volume of depolarized brain is much smaller than the entire brain, so that the geometry of the skull around each electrode can be approximated as a plane.

The first approximation, of separation, corresponds well to bilateral ECT, in which the two electrodes are widely separated and not in the same plane. The approximation that each electrode site can be considered as very small is supported by the observation that small differences in electrode placement cause clinically observable changes. Specifically, it has been reported that moving the two electrodes of symmetrical bifrontemporal ECT forward by just 2.5 cm improved post-ECT cognitive function scores and decreased the variability of these scores, with distinct statistical significance.

Nevertheless, the invention applies to direct current as well as alternating current. The brain volume within a hemisphere of voltage E sufficient to depolarize around the electrode is ⅔ π E³. As voltage rises, this volume increases in proportion to voltage cubed. The volume of brain that is actually depolarized equals the volume within this voltage hemisphere multiplied by the number of voltage carriers, that is the charge. This reveals that for constant voltage ECT instruments, the depolarized brain volume—which represents the stimulus dose—increases with the cube of the voltage multiplied by the charge. This is true whether the ECT stimulus current is bidirectional (alternating) or unidirectional.

This indicates that with bilateral ECT, at the same charge a stimulus of voltage E3 has (E3/E2)³ times the effective (seizure-inducing) dose of a stimulus of voltage E2. Because of safety considerations, modern ECT instruments are typically made to supply constant current rather than constant voltage. The effective seizure-inducing dose of a stimulus can be expressed in terms of current instead of voltage by using Ohm's Law, voltage E equals current I times impedance Z. This reveals that for constant current ECT instruments, the depolarized brain volume—that is, the stimulus dose—increases with the cube of the current multiplied by the charge. This is true whether the ECT stimulus current is bidirectional (alternating) or unidirectional.

This further indicates that, with bilateral ECT, at the same charge a stimulus of current I3 has (I3/I2)³ times the effective (seizure-inducing) dose of a stimulus of current I2. For example, a 900 mA stimulus has 1.42 times the seizure-inducing dose of a 800 mA stimulus at the same charge. Likewise, at the same charge, a 1150 mA current stimulus has 3.0 times the effective dose this charge would have at 800 mA current and 2.1 times the dose it would have at 900 mA current. The direct implication is that the use of an ECT stimulus dose with a current above 900 mA will be more effective at inducing a therapeutic ECT seizure than a stimulus of current in the range of 800 to 900 mA. It also directly implies that the maximum effective ECT stimulus dose that can be achieved within the limitation stipulated by present regulations can be increased by using currents higher than 800 or even 900 mA.

Because it is possible to devise an ECT instrument that delivers stimuli that are not of constant current or constant voltage, it is appropriate to consider more general formulations of stimulus dose that do not require either of these. For such instruments, the stimulus dose according to the present method would be proportional to the time-averaged cube of the voltage multiplied by the charge. An alternative formulation is that it would be proportional to the time-averaged cube of the current multiplied by the charge. Still another alternative formulation would be to combine the current and the voltage so that their total exponent is three, e.g., time-averaged current multiplied by time-averaged square of voltage, or time-averaged voltage multiplied by time-averaged square of current.

The limitation within present U.S. federal regulations is that at 220 Ohms dynamic impedance, the stimulus energy is not greater than 100 Joules. The energy in Joules equals the current in Amps squared multiplied by the dynamic impedance in Ohms and by the current duration in Seconds (I²×Z×t). This also equals the current in Amps multiplied by the product of the charge in Coulombs and the impedance of 220 Ohms. Accordingly, at 800 mA, the maximum charge allowed by U.S. federal regulations is 568 mC, at 900 mA it is 505 mC, and at 1150 mA it is 395 mC. Calculating the maximum dose (in “current-charge dose units”) as the current cubed multiplied by the maximum charge (I³×Q) gives the result that the actual dose is 291 current-charge dose units at 800 mA, 368 current-charge dose units at 900 mA, and 601 current-charge dose units at 1150 mA. That is, the maximum stimulus dose at 1150 mA is 207% of the maximum at 800 mA and 163% of the maximum at 900 mA, clearly substantially larger.

The invention includes an ECT stimulus generator instrument that may incorporate ECT stimuli of current higher than 900 mA, including between 1 A and 2 A. An ECT stimulus generator instrument has been previously described in U.S. Pat. No. 5,871,517, the disclosure of which is hereby incorporated by reference. It includes electrical circuits to transform wall mains current into an electrical stimulus that is applied to the head of the patient. This stimulus consists of a series of bipolar pulses of current that is constant or approximately constant. These pulses are typically of duration in the range of 0.2 to 2.0 msec; the duration of a pulse is called the pulsewidth. For pulsewidths in the range of 0.5 to 2.0 msec, the frequency is typically in the range of 10 to 180 Hz. Because each wave has both a positive phase and a negative phase, this range corresponds to 20 to 360 pulses per second. For pulsewidths in the range of 0.2 to 0.5 msec, the frequency is typically in the range of 15 to 300 Hz. The ECT stimulus generator can include measuring circuits to assure accuracy of the stimulus in regards to dose or any of its components such as charge, current, voltage, dynamic impedance, pulsewidth, frequency, charge rate, or duration. It can include measuring circuits to assure the proper and safe condition of the pathway for the electrical stimulus. The ECT stimulus generator can also include in a single casing (cabinet), the circuitry to amplify electromyogram, electroencephalogram, electrocardiogram, other electrical, auditory, and photovoltaic signals collected from the patient, to filter these signals, to convert them into digital information, to process this digital information as by calculation or sorting, and to display, print, record, or transmit this digital information or the processed information.

The invention is also the instrument that displays the stimulus dose that is calculated as the product of the charge and an exponential power of the current, typically the third power. This display can be in any observable form, such as light emitting display, liquid crystal display, gas plasma display, analog display, cathode ray tube, or paper printout. As explained below, the number of this exponential power can be between 2 and 3 for some electrode placements. Of course all stimulus doses can be multiplied by a constant scaling factor besides unity and this does not change the invention. The invention also includes an ECT stimulus generator that incorporates setting of the stimulus dose by the operator, where this stimulus dose is calculated as the product of the charge and an exponential power of the current, typically the third power but sometimes between two and three.

In the commonly used bifrontotemporal bilateral ECT electrode placement, the two electrodes are well separated and not in the same plane, in accordance with the approximations on which the equations are based. This is true of the bifrontal and left anterior right temporal bilateral electrode placements as well. However, other electrode placements have been used in which the two electrodes are located approximately in the same plane and are not widely separated. One such placement is the Lancaster unilateral placement, with the electrodes over the right temple and just above and behind the right ear. Another is the Abrams and Taylor bianterior placement, with both electrodes on the forehead. When both electrodes are approximately in the same plane and not widely separated there is only a small depth effect, so that the volume of seizure foci increases in two dimensions as the current increases. In these circumstances the stimulus dose is proportional to the charge times the current squared instead of cubed. In the commonly used d'Elia unilateral ECT placement, although the two electrodes are not in the same plane there is little depth between their locations. At the same time the two electrodes are widely separated. Accordingly, the depth effect should be intermediate, and the volume of seizure foci should rise with the current taken to an exponential power between 2 and 3. Accordingly, this exponential power is approximately 2.5. As a first approximation, the stimulus dose for the d'Elia unilateral ECT placement is charge times current to the 2.5 power. Correspondingly, a 900 mA current stimulus has 1.34 times the dose of a 800 mA current and 1150 mA current has 2.5 times the dose of 800 mA current, at the same charge. Accordingly, the invention includes the expression of current to an exponential power of at least 2 and up to 3.

All commercially distributed ECT devices in the USA at this time are believed to use alternating (that is, bidirectional) stimulus current. Each wave is followed by an equal wave in the opposite direction. This means that the net charge passing between the two ECT electrodes is zero, at all stimulus settings. Because it is zero, this net charge is irrelevant to seizure induction. Moreover, brain neurons do not store charge and the mechanism that is part of this invention does not involve their storing charge. All the effects on depolarizable brain neurons of an electrical stimulus wave are not entirely neutralized by the equal next electrical wave in the opposite direction. The effect that lingers from both is that of building towards the development of a brain seizure event. The medical term for this effect is “kindling” and specifically “rapid kindling.”

In the invention, voltage can be used or displayed instead of current. By applying Ohm's Law the stimulus dose can be chosen or displayed as equal to the charge multiplied by the voltage cubed. The concepts are the same. Similarly, a mixture of voltage and current can be used or displayed instead of current. In this, the stimulus dose can be chosen or displayed as equal to the charge multiplied by the product of voltage to an exponential power and current to an exponential power. In this, the voltage might be squared and the current not, or vice-versa, or each taken to the 1.5 power.

1. A method of treating a patient, the method comprising administering an electroconvulsive therapy (ECT) stimulus to the patient, wherein the current is greater than about 0.9 ampere.

2. The method of claim 1, wherein the current is greater than about 1 ampere.

3. The method of claim 1, wherein the current is between about 1.0 and about 2.0 amperes.

4. The method of claim 3, wherein the energy delivered is less than or equal to about 100 Joules across an impedance of 200 ohms.

5. The method of claim 4 wherein the current varies by 0.1 ampere or less.

6. The method of claim 4, wherein the stimulus comprises pulse stimuli having a pulsewidth between about 0.5 and about 2.0 msec.

7. The method of claim 6, wherein the pulse stimuli have a frequency in the range of about 10 to about 180 Hz.

8. The method of claim 4, wherein the stimulus comprises pulse stimuli having a pulsewidth between about 0.2 and about 0.499 msec.

9. The method of claim 8, wherein the pulse stimuli have a frequency in the range of about 15 to about 300 Hz.

10. An apparatus for electroconvulsive therapy comprising an electroconvulsive therapy stimulus generator, wherein the generator is adapted to deliver an electroconvulsive therapy stimulus to the patient, the stimulus comprising a current greater than about 0.9 ampere.

11. The apparatus of claim 10, wherein the generator is adapted to deliver a stimulus comprising a current greater than about 1 ampere.

12. The apparatus of claim 11, wherein the generator is adapted to deliver a stimulus comprising a current between about 1.0 and about 2.0 amperes.

13. The apparatus of claim 12, wherein the generator is further adapted to deliver a stimulus with an energy less than or equal to about 100 Joules across an impedance of 200 ohms.

14. The apparatus of claim 13, wherein the generator is adapted to deliver a current that varies by 0.1 ampere or less.

15. The apparatus of claim 13, wherein the apparatus is adapted to deliver a pulse stimuli having a pulsewidth in the range of about 0.5 to about 2.0 msec and having a frequency in the range of about 10 to about 180 Hz.

16. The apparatus of claim 13, wherein the apparatus is adapted to deliver a pulse stimuli having a pulsewidth in the range of about 0.2 to about 0.499 msec and having a frequency in the range of about 15 to about 300 Hz.

17. A method of calculating the dose of an electroconvulsive therapy stimulus to be delivered to a patient, the method comprising multiplying a charge to be delivered by the current to an exponential power, wherein the exponential power is between about 2 and about 4.

18. The method of claim 17, wherein the exponential power is between about 2 and about 3.

19. The method of claim 18, wherein the exponential power is about 3.

20. A method of calculating the dose of an electroconvulsive therapy stimulus to be delivered to a patient, the method comprising multiplying the charge to be delivered by the voltage to an exponential power, wherein this exponential power is between about 2 and about 4.

21. The apparatus of claim 10, wherein the generator is adapted to deliver a stimulus and to calculate the stimulus as time-averaged n^{th} exponential power of voltage multiplied by the time-averaged m^{th} exponential power of current multiplied by charge, where the sum of the numbers n and m is between about 2 and about 4.

22. The apparatus of claim 12, wherein the generator is adapted to deliver a voltage in the range of about 220 to about 500 volts, and results from the calculation of a current range of 1 A to 2 multiplied by a dynamic impedance in the range of about 150 ohms to about 300 ohms.
